(19) United States
(12) Patent Application Publication
(10) Pub. No.: US 2022/0287651 A1
(43) Pub. Date: Sep. 15, 2022

(54) METHOD AND SYSTEM FOR ANALYZING BIOMECHANICAL ACTIVITY AND EXPOSURE TO A BIOMECHANICAL RISK FACTOR ON A HUMAN SUBJECT IN A CONTEXT OF PHYSICAL ACTIVITY

(71) Applicant: MOTEN TECHNOLOGIES, Giff-Sur Yvette (FR)

(72) Inventor: Maxime Projetti, Giff-Sur Yvette (FR)

(21) Appl. No.: 17/635,223

(22) PCT Filed: Aug. 13, 2020
(86) PCT No.: PCT/FR2020/051465

§ 371 Date: Feb. 14, 2022

(30) Foreign Application Priority Data
Aug. 14, 2019 (FR) 1909206

(51) Int. Cl. A61B 5/00; A61B 5/11

(52) U.S. Cl. CPC A61B 5/7275; A61B 5/1118; A61B 5/1114; A61B 5/486; A61B 5/4519; A61B 5/7264; A61B 5/6801; A61B 5/002; A61B 5/4561; A61B 2503/20; A61B 2562/0219; A61B 2562/0223

(57) ABSTRACT

A method for analyzing the biomechanical activity of a human subject and the exposure to a biomechanical risk factor in a context of physical activity, which comprises collecting signals from sensors of one or more muscles of the subject, collecting signals representing the movement of the subject, and processing these signals to extract signals representative of the vibratory behavior of the muscle or muscles of the subject. This method also comprises detecting a drift of the vibratory signals in relation to a frame of reference of the vibratory behavior of the muscle or muscles in the context of physical activity, and predicting a physiological break time necessary for the subject's muscles to recover their reference vibratory behavior.

### CROSS-REFERENCE TO RELATED APPLICATIONS

This application is a national phase entry under 35 U.S.C. § 371 of International Patent Application PCT/FR2020/051464, filed Aug. 13, 2020, designating the United States of America and published as International Patent Publication WO 2021/028641 A1 on Feb. 18, 2021, which claims the benefit under Article 8 of the Patent Cooperation Treaty to French Patent Application Serial No. 1909206, filed Aug. 14, 2019.

### TECHNICAL FIELD

The present disclosure relates to a method for analyzing the biomechanical activity of a subject and the exposure to a biomechanical risk factor in a context of physical activity. It also relates to a system implementing this method.

The field of the disclosure, in particular, covers physical ergonomics and the ergonomic evaluation of workstations and physical assistance equipment (exoskeletons, cobots, robots).

### BACKGROUND

In physical ergonomics, the methods used are essentially based on analyzing the movement and postures of an operator. The prevention specialist or ergonomist observes the situation and completes rating grids according to criteria specific to each company. Some are based on a dated method called RULA (Right Upper Limb Assessment) widely used for lack of anything better: the observer evaluates the operator's posture at a key instant of the task by assessing the main joint angulations adopted.

It is commonly accepted in the industry that some personnel spend almost all of their time completing this type of grid. Another fundamental limitation of this type of grid is the subjective criterion to obtain a strength score, which may then move the overall score from an acceptable level to a critical level by changing a single rating parameter. The rating must, therefore, be read by an expert and informed eye to obtain a reliable rating. A problem then emerges linked to the need to precisely and objectively quantify the muscular activity of the operator in order to make the ergonomic analysis reliable.

Moreover, the grid is filled in for a limited and short time, thus preventing measurement of the effects of fatigue on the postures of an operator, who can deploy different compensation strategies that are not identifiable with the naked eye. Today, there is no tool to monitor the biomechanical risk factors of an operator in a real situation during an entire work activity.

A final argument concerns the arrival of exoskeletons and other robotic equipment for physical assistance in the new industry 4.0, whose validation from an ergonomic point of view is a real challenge. Indeed, an exoskeleton can adopt an acceptable posture from the point of view of the RULA method while overloading certain muscle groups, which may then cause accidents or occupational disease in the long term.

Furthermore, monitoring the muscular activity of the human body is an important function in many applications linked to health, sports and robotics. Characterizing the state of a muscle (such as fatigue), and the evolution of this state according to the movements of the whole body, may provide valuable information on the condition of the muscle in order, for example, to optimize the training in an athlete or adapt the physical workload of an industrial operator. In addition, recognizing the signs of muscle fatigue helps prevent the risk of injury related to physical activity in a sporting or industrial context.

The muscular activity is generally captured by electromyography (EMG) [1], i.e., by measuring the electrical activity of the muscle. EMG techniques may be invasive with the insertion of electrodes directly into the body of the muscle, or superficial by the use of surface electrodes adhered to the skin. The latter technique has many drawbacks and its use is limited to controlled environments such as laboratories. It requires the use of single-use electrodes for hygiene reasons and requires the use of conductive gel to ensure good coupling with the skin and thus to limit impedance changes in the event of perspiration. In addition, technical know-how is necessary to position and use the electrodes. This, therefore, imposes severe limitations on the practicality of such an out-of-laboratory measurement.

So-called mechanomyographic (MMG) methods involving the measurement of micro-vibrations induced during muscle contractions remove a large number of limitations imposed by EMGs: immune to perspiration and, therefore, to changes in impedance on contact with the skin, better signal to noise ratio (SNR) and less sensitivity to positioning at the muscle to be analyzed. The notion of “listening to the sounds” produced by the muscles on the surface of the skin dates back to the early 1800s [2]. This method is characterized by the capture and the interpretation of the mechanical vibratory activity during muscle contractions.

The vibrational activity is produced by the lateral oscillations of the muscle fibers, which occur at the resonant frequency of the muscle [3]. The MMG signal, of a low-frequency nature (2-250 Hz), is obtained by means of accelerometers, microphones, piezoelectric sensors or even by laser. Scientific research shows that analyzing MMG signals allows the examination of many characteristics of muscle function such as neuromuscular fatigue [4], the efficiency of anesthesia [5], or certain neuromuscular syndromes such as Parkinson's disease [6].

Regarding terminology, there are different names to describe mechanomyography [7]: acoustic or phonic myography (AMG, PMG) and vibrational myography (VMG). Typically, acoustic myography uses pressure sensors, microphones or piezoelectric transducers, while vibration myography uses accelerometers almost exclusively. The preferred device of this disclosure uses MEMS capacitive accelerometers, but may be extended to the other sensors mentioned.

The instruments used to capture movements and postures generally use inertial units composed of three-axis accelerometers, three-axis gyrometers and three-axis magnetometers. The usual designations of inertial units are acronyms derived from English: Inertial Reference System (IRS), Inertial Navigation System (INS), or Inertial Measurement Unit (IMU). This last designation concerns only the sensor block (accelerometer, gyrometer and magnetometer), without a calculation unit. These systems are found in a large number of connected objects owing, in particular, to MEMS technology, which has enabled the miniaturization of components. In addition, technological developments in batteries and wireless communication have made these systems ultra-portable with significant autonomy. However, the use of inertial units for biomechanical analyses outside the laboratory is faced with a significant obstacle: the presence of magnetic fields generating distortions and causing the measurement of the magnetometers to drift [8]. This drift is reflected in the movement data, which then becomes unusable. Another disadvantage for this type of system is the prior calibration of the sensors to align them with the segments of the body to be analyzed.

The instruments used to capture movements and postures may include optical systems without markers with, in particular, the emergence of so-called depth cameras. They allow resolution of the ambiguities inherent in monocular systems (segmentation, auto-occultations and ambiguities induced by planar projection) by directly providing depth images of the scene to estimate a person's posture. Another advantage of this type of camera is that the 3D information of the scene is provided from a single point of view [9]. A limitation of depth cameras comes from their range of less than 5 m. This limitation may be lifted by using several cameras, but a restrictive calibration or a very controlled environment then becomes necessary.

An understanding of muscular activity in correlation with whole-body movements is, therefore, critical in contexts with high biomechanical stress outside of controlled environments. The example of musculoskeletal disorders (MSDs) in industry is outlined. Indeed, epidemiological studies show that MSDs result from exposure to the following biomechanical factors:
Postural constraints
Efforts and the dynamic force mobilized
Static muscular work
Repeated movements over a long period of activity.

These biomechanical stresses are evaluated using three criteria: the intensity of the stress, the frequency of exposure to this stress and the duration of exposure. In addition, certain environmental factors may aggravate these biomechanical factors: mechanical pressures, shocks and impacts, vibrations and thermal environments.

The techniques used in ergonomic studies to analyze the biomechanical activity of a subject and the exposure to the risk factors mentioned above are mainly based on observations by an expert (ergonomist) and on photo or video analysis. In addition to the subjective nature of such an analysis, it is not possible as it stands to have real-time follow-up of the subject over very long periods. Recently the introduction of motion capture instruments has been observed, such as inertial units worn on the body segments to be analyzed, or depth cameras. However, these techniques are limited by nature to movements and postures and neglect the measurement of the muscular activity induced by all the movements of the body.

The combination of EMG electrodes, MMG sensors and IMU sensors has already been the subject of several patents for various applications.

Thus, document US20130317648A1 [10] relates to a sleeve integrating an array of EMG electrodes and an IMU unit for movement recognition intended for to control machines or robotic systems.

Document US20170312576A1 [11] also deals with a sleeve integrating an array of EMG sensors and an IMU unit for sports training or therapeutic use.

Documents US20150169074 [12] and US20140240103 [13] from Thalmic Labs deal with a connected strip containing EMG electrodes, an MMG accelerometer and an IMU unit for movement recognition to control connected objects, for example, connected glasses.

Document WO2015/063520 A1 [14] merges movement data obtained by an IMU unit with biomechanical data from an MMG or EMG sensor for applications such as: patient rehabilitation, classification of postures and gestures, or even monitoring the health of a fetus.

Document US 20110196262 A1 [15] provides an exhaustive methodology for processing data from a MEMS accelerometer (1 or 2 axes) in order to quantify absolute muscular effort in real time. The field of applications covers the analysis of sports activity and patient rehabilitation.

Document U.S. Pat. No. 10,292,647 B1 discloses a wearable device comprising a contraction sensor and a motion sensor and transmitting signals to a processor that analyzes the signals. The contraction signal determines whether the user's muscle is contracted or relaxed. Contraction and movement data is sent to and viewed on the smart device screen along with video of the user performing a movement. Simultaneous viewing of video and sensor data provides immediate feedback to the user regarding the timing of trunk contractions with body movements in an athletic, training, or therapeutic movement to allow that user to modify and improve the coordination of trunk contraction with body movements to improve movement performance and achieve better results.

However, these systems do not allow an analysis of the vibratory behavior of a muscle of a subject aimed at producing indicators of muscular fatigue from portable and autonomous devices.

It is particularly necessary to propose new methods and instruments for the objective, reliable and precise rating of workstations or physical assistance equipment. These methods must be digitized for a massive deployment and take into account a key parameter, insufficiently characterized to date: muscular activity.

The object of the present disclosure is to remedy these drawbacks by proposing a new method for identifying biomechanical risks in an uncontrolled environment based on objective measures of biomechanical factors of a subject, the degree and the frequency of exposure of this subject to these same factors. Automating this method via ultra-portable instrumentation, communicating the biomechanical data wirelessly, over long acquisition periods is also the objective of the disclosure.

### BRIEF SUMMARY

This objective is achieved with a system for analyzing the biomechanical activity of a human subject and the exposure to a biomechanical risk factor in a context of physical activity, comprising:
a. means for collecting vibratory signals, attached to one or more first body segments of the subject, the measurement of which reflects local muscular activity;
b. means for collecting signals representing the movement of the subject, the measurement of which reflects the orientation and the movement of one or more second body segments in 2 or 3 dimensions;
c. means for processing these signals so as to extract therefrom indicators representative of the intensity of the biomechanical stress,

According to the disclosure, this analysis system further comprises:
d. means for detecting a drift of the vibratory signals with respect to a frame of reference of the vibratory behavior of the muscle(s) in the context of physical activity,
e. means for predicting a physiological break time necessary for the muscles of the subject to recover their reference vibratory behavior.

The disclosure also relates to a method for analyzing the biomechanical activity of a human subject subjected to physical exercise, and the exposure to one or more biomechanical risk factors such as:
Postural constraints
Efforts and the dynamic force mobilized
Static muscular work
Repeated movements over a long period of activity.

This analysis method uses a measurement system, focused on one or a multitude of body segments of interest, in a context of deployment in an uncontrolled environment, comprising:
a. collecting vibratory signals by a vibration sensor, attached to one or more first body segments of the subject, the measurement of which reflects the local muscular activity;
b. collecting signals representing the movement of the subject, the measurement of which reflects the orientation and the movement of one or more second body segments in 2 or 3 dimensions;
c. processing these signals to extract indicators representative of the intensity of the biomechanical stress, this processing implementing data merging techniques.

According to the disclosure, this method further comprises:
d. detecting a drift of the vibratory signals with respect to a frame of reference of the vibratory behavior of the muscle(s) in the context of physical activity,
e. predicting a physiological break time necessary for the muscles of the subject to recover their reference vibratory behavior.

Merging MMG and IMU data is likely to bring another dimension to the analysis of human activity by creating a bridge between movement dynamics and muscular activity. Taking patient rehabilitation as an example, the recovery of joint mobility is based solely on the range of motion that the patient can produce over the instant. The muscular activity information may provide critical information on recovery progress from a physiological and biomechanical perspective. However, as mentioned above, these muscular activity tests are carried out exclusively by EMG, in controlled environments and over very short times.

The use of movement and muscular activity data merging techniques applied to physical ergonomics leads to the development of algorithms to automatically segment human movement using IMU data and to associate a physiological impact via MMG data. From then on, it becomes possible to characterize the mechanical efficiency of the professional gesture and the mechanisms of loss of this efficiency in relation to fatigue.

In a particular embodiment of the disclosure, the identification method further comprises collecting the context or the scene in which the human subject is evolving by optical collection means, and processing this context or scene, so as to generate information on the posture and the gesture of the human subject in correlation with the vibratory behavior signals.

The posture information may be used to constitute a classification of movements in a predetermined frame of reference.

The identification method according to the disclosure may also comprise a cross-analysis of the movement and muscular activity signals so as to deliver information on the performance and the health of the human subject during a physical activity in a work or sports activity context.

It may also comprise merging movement data and muscular activity data so as to recommend a personalized arrangement of physiological breaks so that the muscular tissues of the human subject return to their rested state in the metabolic and mechanical sense after an effort.

The means for collecting movement signals may advantageously comprise an inertial unit IMU (Inertial Measurement Unit) using MEMS technology.

The inertial unit IMU may be integrated together with the muscular activity sensor means to obtain co-localized measurements at a body segment of the human subject.

The inertial unit IMU may be of the six-axis type measuring rectilinear accelerations (three axes) and rotations (three axes).

The means for collecting the movement signals may also comprise a magnetometer (three axes) to determine the orientation of the body segment with respect to the Earth's magnetic North.

The means for sensing muscular activity may advantageously comprise an MMG (mechanomyographic) accelerometer arranged to generate a mechanomyographic signal. This sensor is ideally a high-performance capacitive MEMS accelerometer derived from seismic prospecting [16].

The analysis system according to the disclosure may implement a plurality of measurement nodes firmly attached to body segments of a human subject. Each measurement node comprises means of communication with a receiving station that implement a communication protocol of the Bluetooth Low Energy (BLE) type.

### BRIEF DESCRIPTION OF THE FIGURES

The disclosure will be better understood with reference to the following figures.

FIG. 1 illustrates the principle of the method according to the disclosure, producing information on the degree of exposure to biomechanical risk factors.

FIG. 2 illustrates an architecture of a measurement node implemented in the method according to the disclosure.

FIG. 3 is a block diagram illustrating the operations carried out by the method according to the disclosure.

FIG. 4 is an example of data recovery by the analysis method according to the disclosure.

FIG. 5 is an illustration of another type of result produced by the device: the muscular effort and its drift over time are measured in real time during the activity of the person and the necessary break time is calculated to avoid injuries and prevent MSD risks (according to Baillargeon [17]).

FIG. 6 is an illustration of the method for calculating a physiological break time from the processed data of the device. The upper curve shows the revolution of the RMS amplitude obtained from the filtered mechanomyographic MMG signal, and reflecting the intensity of the biomechanical stress. The lower curve shows the mean power frequency MPF obtained after processing the mechanomyographic signal in the frequency domain. This curve reflects the muscle fiber recruitment strategy.

FIG. 7 illustrates an application of the disclosure for the ergonomic evaluation of work situations and equipment used by industrial operators. In particular, the method according to the disclosure is used for the ergonomic analysis of an exoskeleton supporting portable electrical equipment.

FIG. 8 shows typical signals collected from industrial operators in the activity presented in FIG. 7. The upper curve presents the angular amplitudes in flexion/extension of the right shoulder, while the lower curve presents the muscular vibrations of the right bicep associated with the movements produced.

FIG. 9 shows analysis results of the impact of an exoskeleton supporting a jackhammer on the right shoulder of an operator.

FIG. 10 illustrates another application of the disclosure for the sports field. A runner is equipped with the measurement system according to the disclosure at the right vastus lateralis.

FIG. 11 shows the typical signals collected during a repetitive stride by a runner. The upper curve presents the angular amplitudes in flexion/extension of the right hip, while the lower curve presents the muscular vibrations of the right vastus lateralis associated with the movements produced.

FIG. 12 shows a time-frequency representation of the stride in a runner from the raw signals of FIG. 11. The x-axis shows the time. Each vertical line represents a stride cycle with its frequency signature, that is to say, the distribution of the energy of the signal over the frequency band of interest.

FIG. 13 illustrates a specific example of the implementation of a method for obtaining a quality mechanomyographic (MMG) signal used for the present disclosure.

FIG. 14 illustrates a particular example embodiment of a step for extracting muscular activity parameters.

### DETAILED DESCRIPTION

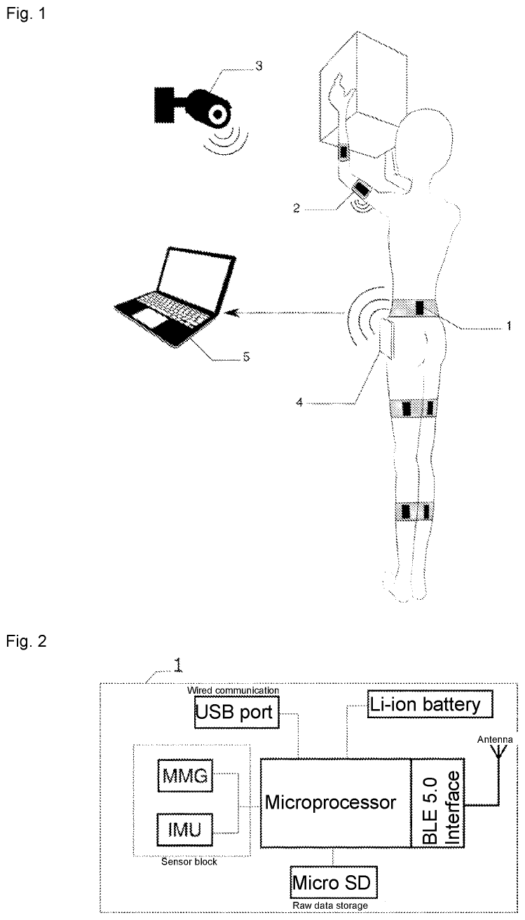

The principle is illustrated in FIG. 1: the preferred use of the device integrates the IMU sensor and the muscular activity sensor in the same box or measurement system 1. However, these two sensors may be separated. Moreover, the disclosure also includes an integration of the components directly within a garment, for example, in the form of a compression band 2 serving to hold the sensor on the body segment, thus conferring a good mechanical coupling to detect muscle vibrations and to limit measurement artifacts.

Furthermore, the method according to the disclosure also extends to measurements of postures and movements using optical systems without markers, such as depth cameras 3. This may prove to be an interesting alternative to IMU sensors in an uncontrolled environment with a restricted movement space, or may even offer redundancy in the measurements in order to validate the postures and movements of the body while providing the elements of context in which the subject evolves (obstacles, objects, etc.).

Embedded signal processing electronics allow certain calculation operations to be executed in order to facilitate wireless communication to an external receiver 4 (smartphone type). This receiver may then perform complex operations on the data and/or communicate them to a computer/server 5 via a mobile data network. The cross-analysis of movement and muscular activity data provides key information on a person's performance and health in their daily activity. Merging movement and muscular activity data, in particular, allows a personalized arrangement of physiological breaks so that the muscle tissues return to their rested state in the metabolic and mechanical sense after an effort.

A description will now be given of measurement systems implemented in a practical embodiment of the disclosure. To measure movements and postures, a distinction is made between instruments for detecting movements and postures. Three categories have been identified: commercially available IMU sensor, MEMS sensitive element for integration into a connected device (clothing or object) and depth cameras.

The external IMU sensor may be chosen as a fully integrated IMU sensor for 3D motion capture, for example, the MTw Awinda motion tracker [18] from the company XSENS. The specifications of this sensor are summarized in Table 1:

TABLE 1
Xsens MTw Awinda Device Specifications

| Characteristic | Value | |
|---|---|---|
| Full-scale 3D accelerometer | ±1.6 | g |
| Full-scale 3D gyrometer | ±2000°/s | |
| Full-scale 3D magnetometer | ±190 | μT |
| Static resolution (pitch/roll) | 0.5° | RMS |
| Static resolution (yaw) | 1° | RMS |
| Dynamic resolution (pitch/roll) | 0.75° | RMS |
| Dynamic resolution (yaw) | 1.5° | RMS |

A MEMS technology inertial unit IMU may be integrated together with the muscular activity sensor to obtain co-localized measurements at a given body segment. This unit may be six axes measuring rectilinear accelerations (three axes) and rotations (three axes). It is also possible to associate a magnetometer (three axes) to determine the orientation of the body segment with respect to the Earth's magnetic North. Components from Invensense were selected for their performance and cost. Their characteristics are summarized in Table 2:

TABLE 2
Specifications of two MEMS inertial units

| | | |
|---|---|---|
| Characteristic | ICM-20649 | ICM-20948 |

| | | |
|---|---|---|
| Number of axes | 6 | 9 |

| | | | | |
|---|---|---|---|---|
| Full-scale 3D accelerometer | ±4/8/16/30 | g | ±2/4/8/16 | g |
| Full-scale 3D gyrometer | ±0.5/1/2/4 | k °/s | ±0.25/0.5/1/2 | k °/s |

| | | | |
|---|---|---|---|
| Full-scale 3D magnetometer | — | 4900 | μT |

| | | | | |
|---|---|---|---|---|
| Resolution | 16 | bit | 16 | bit |

| | | |
|---|---|---|
| Internal noises | 285 μg/√Hz | 230 μg/√Hz |
| | 17.5 m°/s/√Hz | 15.5 m°/s/√Hz |

The Microsoft® Kinect® is a low-cost system consisting of a color camera (RGB), an infrared camera and an infrared projector. This system was used to capture the movements and postures of industrial operators by Plantard in [9]. The characteristics of the Kinect V1 and V2 are presented in Table 3:

TABLE 3
Specifications of the Kinect V1 and V2

| Characteristic | V1 | | V2 | |
|---|---|---|---|---|
| Acquisition | 30 | Hz | 30 | Hz |
| frequency | | | | |
| Range | [0.8-4] | m | [0.8-4] | m |
| Field of view | 57 × 43 | [deg × deg] | 70 × 60 | [deg × deg] |
| Color resolution | 640 × 480 | [pix × pix] | 1920 × 1080 | [pix × pix] |
| Depth resolution | 320 × 240 | [pix × pix] | 512 × 424 | [pix × pix] |

To measure muscular activity, the constant components of a three-axis accelerometer are eliminated in order to retain only the variations due to micro-vibrations on the surface of the skin. The sensor requires a very low noise floor of less than 100 μg√Hz in order to capture these phenomena. Accelerometers used in seismic prospecting are good candidates for measuring mechanomyographic signals. Two preferred components for this disclosure have been selected: the ADXL354 and its digital equivalent ADXL355 [19], with their performance summarized in Table 4.

TABLE 4
Specifications of Analog Devices Accelerometers
for Mechanomyographic Measurement

| Characteristic | ADXL 354 | ADXL 355 |
|---|---|---|
| Type of output | Analog | Digital |
| Noise floor | 20 μg/√Hz | 25 μg/√Hz |
| Sensitivity | 400/200/100 mV/g | 3.9/7.8/15.6 μg/LSB |

| | |
|---|---|
| Full scale | ±2/4/8 |
| Bandwidth | 1000 Hz |

The preferred use of the device integrates the IMU sensor and the MMG sensor in the same box. However, these two sensors may be separated, with one system for measuring postures and another system for measuring muscular activity. In addition, it is possible to provide integration of the components directly within a connected garment (for example, a compression band serving to hold the sensor in position on the body segment). Furthermore, the system according to the disclosure also extends to measurements of postures and movements using optical systems without markers, such as depth cameras. This may prove to be an interesting alternative to IMU sensors in situations where the workstations are part of a well-defined environment, or to provide information on the context of the scene and redundancy in the measurements in order to validate body postures and movements.

Electronics embedded in the sensors allow certain calculation operations to be performed in order to facilitate wireless communication to an external receiver 4 (for example, a smartphone or a data collector). This receiver may then perform complex operations on the data (synchronization, segmentation, processing) and communicate analysis results to a computer 5, a smartphone or a cloud.

The results produced, for example, concern the level of exposure to certain biomechanical risk factors (postures, intensity of muscular activity) and the monitoring of these factors during physical activity. With reference to FIG. 4, alerts may potentially be produced to warn the person when he is exposed to a demanding situation from a biomechanical point of view:
Too strong: intense muscular activity obtained via the mechanomyographic signal
Too fast: high movement speed obtained via the speed and the acceleration of the body segments
Too far: obtained via joint angulations
Too long a time: the segmentation of the data allows the exposure time to be calculated

An embodiment of this disclosure provides a second level of analysis after post-processing of the data. The movement and muscular activity data are processed on a PC by an algorithm allowing calculation of the biomechanical performance of the subject's gestures and characterization of their physiological and biomechanical impact on the body. Another result provided by merging the data is the calculation of a physiological break time so that the subject's muscles recover their reference vibratory behavior, thus avoiding exposure to the risk of accidents or occupational diseases.

A few examples of the production of essential technological bricks will now be described. Thus, for the MMG sensor, one may provide:
a MEMS accelerometer meeting the performance requirements for measuring MMG signals.
signal processing tools to extract MMG parameters related to muscular effort and fatigue and filter measurement artifacts.

The motion sensor may integrate:
a MEMS inertial unit 9D for joint integration with the MMG sensor.
inertial data merging algorithms to correct sensor drift and sensitivity thereof to stray magnetic fields (use of a Kalman filter).
a calibration method that is robust over time and quick to set up so as to disturb the operator and the production context as little as possible. This calibration must precisely synchronize all of the deployed sensors and guarantee reliable movement signals.

The data collector (receiver) may integrate:
functions of reception, time synchronization of movement and mechanomyographic data using an external clock (smartphone clock, timestamp sent by PC, etc.)
data storage in a source file for post-processing on a PC;
an algorithm for automatic segmentation of movements into a series of sequences.

The merging of inertial and mechanomyographic data may include:
calculating the level of exposure to biomechanical risk factors and monitoring these factors during physical activity.
an effort model to convert the mechanomyographic signal into a force signal using calibrated effort data for each muscle group of interest.
an algorithm for calculating biomechanical performance from source file data, including synchronized motion and mechanomyographic sensor data.
implementing machine learning algorithms with the mission of automatically recognizing operator gestures and their impact on the musculoskeletal system.
calculating a physiological break time so that the subject's muscles recover their reference vibratory behavior

The following paragraphs describe signal processing tools implemented in the method according to the disclosure. Using orientation measurement merging algorithms is necessary so as to have access to the movement and posture parameters. Typically, these algorithms rely on a Kalman filter as presented in [20].

To condition the mechanomyographic signal, in laboratory conditions, the researchers generally over-sampled the signals with a frequency of the order of 1 kHz or 2 kHz, while the characteristic frequencies of the MMG signal are below 250 Hz. With a view to deployment in the field with wireless data communication, a compromise between the volume of data to be transmitted and sampling was found by setting the sampling frequency to 500 Hz according to the Nyquist criterion. The raw signals from the accelerometer are digitized and then conditioned. The digitized MMG signal has two components: a static component (DC) and a dynamic component (AC). The DC component is not useful for the evaluation of muscular activity and must, therefore, be filtered. Moreover, the movements of the body are low-frequency components that pollute the muscular activity information. In fact, the cutoff frequency of the high-pass filter is comprised in a band between 2 Hz and 50 Hz with a preference for 20 Hz in order to clean the parasitic components mentioned above.

Applying a low-pass filter cuts high-frequency noise and limits the band of interest to frequencies that are characteristic of muscle micro-contractions. A cut between 70 Hz and 250 Hz, more particularly between 200 Hz and 250 Hz, is ideal for analyzing the MMU signal. A preferred value of 250 Hz has been established.

Using a Butterworth low-pass digital filter or a Savitzky-Golay filter are common practices in today's art.

For filtering operations, a five-pole Butterworth filter is ideal due to its constant gain across its passband despite lower roll-off compared to Chebyshev or elliptical filters. In addition, the ADXL355 digital accelerometer offers programmable low-pass and high-pass filters to select the frequency band of interest.

The processing of the mechanomyographic signal relies on the same developments as its electromyographic counterpart. The methods may be divided into four groups: temporal and frequency methods (the most traditional), then time-frequency and time-scale methods (more recent). The choice of an appropriate processing method is then crucial for the objective analysis of MMG signals. Indeed, during an isometric contraction (contraction of a muscle without change in muscle length), the signal may be assumed to be stationary (i.e., its statistical properties are invariant over time) and the conventional signal processing methods based on the Fourier Transform are, therefore, applicable. However, during movements with variable dynamics, a muscle may change its length or recruit more motor units, thus giving rise to so-called non-stationary signals. In this type of muscular activity, the use of time-frequency or time-scale methods becomes necessary. Some parameters extracted from [21] are presented below with particular attention paid to their use and their limits.

Once the mechanomyographic signal has been segmented and properly conditioned, the parameters of interest can finally be extracted. The MMG signal has three components (MMGX, MMGY and MMGZ), representing the accelerations induced by the vibrations of the muscle fibers along the three spatial directions (X, Y, Z). A “total” acceleration signal is calculated by the following operation:

∥MMG∥=\sqrt{MMG_{X}²+MMG_{Y}²+MMG_{Z}²}

The RMS (Root Mean Square) amplitude of the “total” MMG signal then makes it possible to obtain information on the force developed by a muscle. The RMS amplitude varies with fluctuations in muscle fiber tension and increases with the level of muscle contraction. It is the most used parameter in a temporal analysis of the MMG signal and is obtained by the following formula:

$$RMS = \sqrt{\frac{1}{N} {\sum }_{i = 1}^{N} {‖ MMG ‖}_{i}^{2}}$$

With N the observation window equal to the characteristic period of the motion sequence divided by 2. This characteristic period is defined by a cycle of the movement studied, such as, for example, a step in the case of walking, a stride in the case of running, or even the period of handling an object. In the case of static postures, a window of 1 second allows an RMS amplitude to be established comprising sufficient attributes on the physiological and biomechanical behavior of the underlying muscles.

However, the sensitivity of this parameter to physiological tremors and other mechanical artifacts requires additional analysis methods.

The analysis of the power spectral density (PSD) of the MMG signal allows observation of the fluctuations of the frequency content in order to deduce information on muscle fatigue. The standard tool for this type of analysis is the Fast Fourier Transform (FFT) to go from the time domain to the frequency domain. Parametric methods using auto-regressive (AR) models allow estimation of the PSD of the MMG signal without using apodization windows and thus provide better resolution. The most common methods are: Yule-Walker and Burg. The preferred PSD method in this disclosure is that of Yule-Walker. Once the PSD has been estimated, the mean frequency (MPF for Mean Power Frequency) may be determined by the following formula:

$$MPF = \frac{{\int }_{0}^{{f}_{s} / 2} f \cdot PSD ( f ) df}{{\int }_{0}^{{f}_{s} / 2} PSD ( f )}$$

with PSD in g²/Hz, the power spectral density of the MMG signal and fs in Hz, the sampling frequency. MPF is an important metric for examining changes in muscle condition and detecting characteristic signs of fatigue.

During activities with variable dynamics, muscles may change their length, recruit more motor units and adapt the frequency of stimuli, conferring a non-stationary behavior to the MMG signal. Time-frequency approaches are, therefore, necessary to segment the signal in the time domain before performing a frequency analysis. A compromise between ease of execution on a microcontroller and battery conservation (for wireless communication) is the local Fourier transform (STFT for Short Time Fourier Transform), in which a window “slides” over the time signal and allows the PSD to be obtained at a given instant

$${S}_{MMG} ( τ , f ) = {\int }_{- ∞}^{+ ∞} x ( t ) h ( t - τ ) {e}^{- 2 j π f t} dt$$

with x(t) the MMG signal, h(t−σ) the sliding window and σ the parameter allowing the information to be analyzed spectrally at all times.

The disadvantage of such a method is the selection of an adequate data range, which can introduce a resolution defect in the frequency domain. One of the innovative characteristics of this disclosure resides in the use of the orientation measurements of the IMU sensor to segment the MMG signal in an appropriate manner.

Other time-frequency methods, such as the wavelet transform (WT for wavelet transform) and the Wigner-Ville transform (WVT), are frequently used in the laboratory or other controlled environments to analyze MMG signals.

More recent methods called time-scale methods have met with great success with scientists for the processing of MMG signals. One method used, in particular, in McLeod's disclosure [15] is wavelet packet analysis (WPA). This differs from other time-frequency methods because of the multi-scale decompositions of the starting signal, which are separated into low-frequency coefficients (levels of approximation) and high-frequency coefficients (levels of detail). These coefficients then form a “wavelet packet.” A specific toolbox is available in the MATLAB® software.

This method is very efficient for the analysis of MMG signals, but requires heavy post-processing and does not lend itself to automating the estimation of muscle fatigue in real time. Furthermore, complex calculation operations are necessary, which seems incompatible with integration within a connected object that must communicate wirelessly for several hours, and in uncontrolled environments. The present disclosure nevertheless allows a multi-resolution analysis based on the maximum overlap wavelet decomposition (Maximum Overlap Discrete Wavelet Transform: MODWT) as part of the post-processing of raw MMG data. This technique allows the extraction of movement artefacts from the muscle signal with greater precision than with a conventional filtering technique.

The following paragraphs describe procedures for the method according to the disclosure.

A measurement system 1, the architecture of which is presented in FIG. 2, is positioned on one or more body segments of a person in order to estimate the level of biomechanical stress during his activity. Each node is firmly attached to the body segment by means of elastic bands, slightly compressing the sensor on the skin and thus conferring a good mechanical coupling to detect muscle vibrations and limit measurement artifacts.

Depending on the complexity of the task and the number of muscles used, the user may equip himself with one or more nodes that he positions on the belly of each muscle to be analyzed. Each node may communicate information to and/or receive information from a receiving station (e.g., a smartphone). The communication protocol chosen for this disclosure uses Bluetooth Low Energy (BLE). The advantages of BLE are reduced energy consumption and allowing the slave device to remain “discoverable” by a master device while minimizing its consumption. Similarly, a slave device may remain connected to a master device and exchange data at periodic instants. In the case of BLE, there is no limitation on the number of peripherals supported by the same master, in contrast to conventional Bluetooth limited to seven peripherals. The standard gross throughput in BLE is 1 Mbps theoretical, but remains capped at 250 kbps in practice, to be shared between all the slave nodes. A characteristic of the method according to the disclosure is to allow an analysis of the various biomechanical risk factors, by causing a multitude of sensors to communicate simultaneously while supporting a usage range of 8 hours. The data are also stored in a micro-SD-type memory. The measurement node supports wired communication via a USB port.

The receiving station, through near field communication (NFC), is able to activate the sensors and associate a body position with them. Detecting the position of the sensor on the body allows certain signal acquisition parameters to be adjusted, such as the template of certain filters. Once the sensors have been installed and marked, the acquisition may then begin with a simple command at the receiving base. The raw motion data from the IMU sensor will then be processed and merged by the microcontroller of the measurement node in order to send information on the orientations and positions of each segment and joint to the receiving base. The internal processing of the motion data allows the output signal to be sub-sampled in order to optimize battery consumption and the volume of information to be transmitted. Sampling of IMU output data is typically between 50 Hz and 120 Hz. The receiving station then performs calculation operations: counting and detection of excessive movement amplitudes, etc. Alerts on repeated postures and movements may be sent to an external computer or produced on a smartphone.

In order to obtain redundancies in the movement data, or in the absence of IMU sensors, using depth cameras or other optical systems without markers allows access to the movement data necessary for the biomechanical analysis and also allows collection of the context elements of the scene.

In addition to producing results relating to constraining movements and postures, the MMG sensor, integrated into the device, offers indicators on the force deployed by the muscles and muscle fatigue as well as the distribution of the stresses on all of the body segments analyzed.

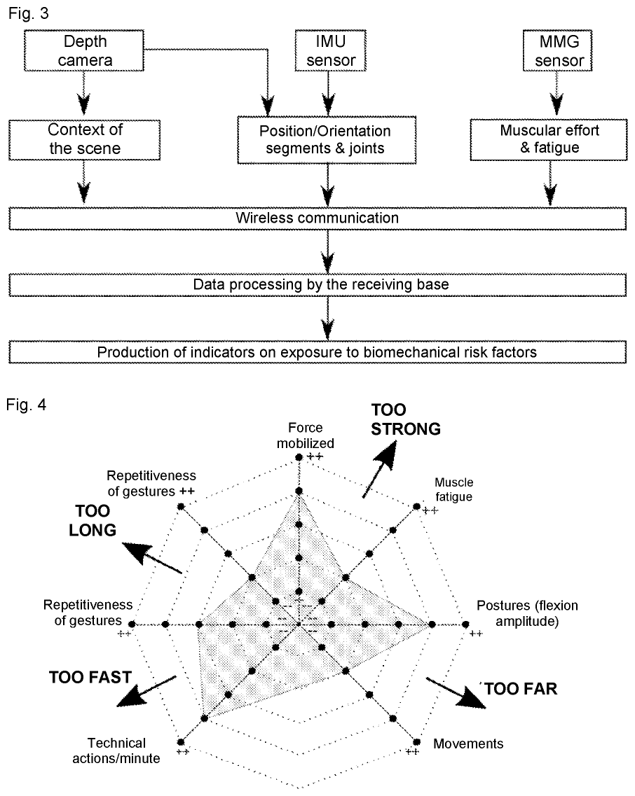

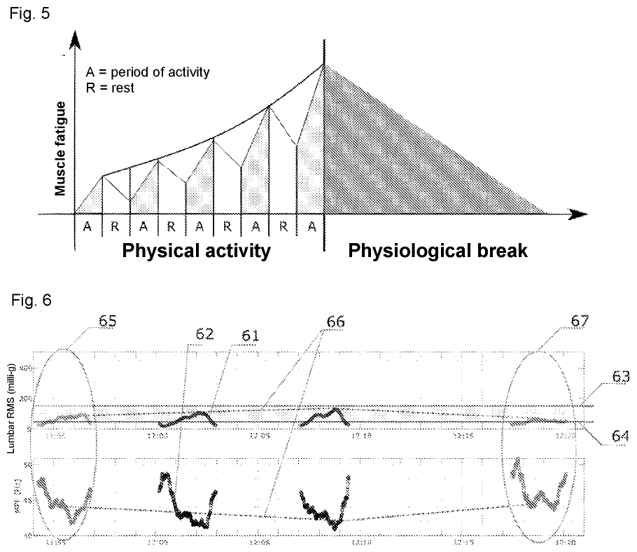

Cross-analysis of movements and mechanomyographic signals allows objective quantification of the level of biomechanical stresses on a human subject. A block diagram describing all the operations and analyses carried out by the method according to the disclosure is proposed in FIG. 3. In this operating mode, the IMU sensor characterizes certain parameters such as the number of technical gestures per minute, the speed of the gestures and the joint angulations. Furthermore, the vibratory signals from the MMG sensor are broken down into segments of 1 second duration at which the RMS amplitude level and the average frequency of the MPF power spectrum are calculated. It is commonly accepted in the art that the RMS amplitude of the MMG signal reflects the level of force deployed by the muscles. However, analyzing the MPF makes it possible to highlight changes in terms of muscle activation (muscle fiber recruitment strategy, muscle fatigue, etc.), which allows a fatigue index to be given. An example of biomechanical risk factor recovery is shown in FIG. 4. In addition, the drifts over time of these biomechanical risk factors may be analyzed by linear regression in order to calculate the linear regression coefficient (Pearson method). This indicator is useful for extrapolating the level of physical strain over time and predicting the physiological break time necessary for the subject's muscles to recover their reference mechanical state (see FIG. 5, according to Baillargeon [17]).

In a particular mode of operation relating to preventing risks linked to biomechanical stresses, take the example of a person performing a repeated activity, such as mobilizing his lower back. The method of the present disclosure as well as the associated measurement system may be used to calculate the break time necessary for the muscles of the lumbar zone to return to a reference state, corresponding to a low level of physical strain, illustrated in FIG. 6. The level of muscular activity is characterized by the RMS amplitude 61 obtained from the filtered mechanomyographic MMG signal. It is associated with the mean power frequency MPF 62, the variations of which indicate the change in the muscle fiber recruitment strategy. A maximum strain threshold 63 is first determined, taken, for example, at 30% of the maximum value of the filtered mechanomyographic signal. Other techniques for determining this strain threshold may be envisaged by taking, for example, the maximum voluntary force (MVF) at the muscle group of interest. A minimum strain threshold 64 is then determined, taken, for example, at 10% of the maximum value of the filtered mechanomyographic signal. The objective is to calculate the break time to bring the level of physical strain on the lumbar muscles toward the minimum threshold.

A mean value of the RMS amplitude and of the mean power frequency MPF is calculated for the segment of interest 65. The calculation is repeated for each activity segment and a linear interpolation is performed for the mean RMS amplitude and mean MPF 66. It is noted that the RMS profile increases during the activity, demonstrating an increasingly demanding activity for the lumbar muscles, as well as a reduction in the MPF, demonstrating the recruitment of a greater number of muscle fibers.

In order to calculate a physiological break time for the muscles to return to their reference state, the technique used in the present disclosure uses the linear regression coefficient calculated previously to determine a linear decrease profile of the mean RMS amplitude toward the minimum physical strain threshold. Conversely, a linear growth profile is determined to bring the MPF back to a reference state, called non-fatigued.

By comparing to a reference state, it is, therefore, possible to determine variations or drifts of the fatigue index (MPF) and the force index (RMS amplitude), indicating the change in the muscle fiber recruitment strategy and the stress intensity, respectively. An alert may be sent to the operator to warn him of his exposure in a zone of significant physical and biomechanical strain.

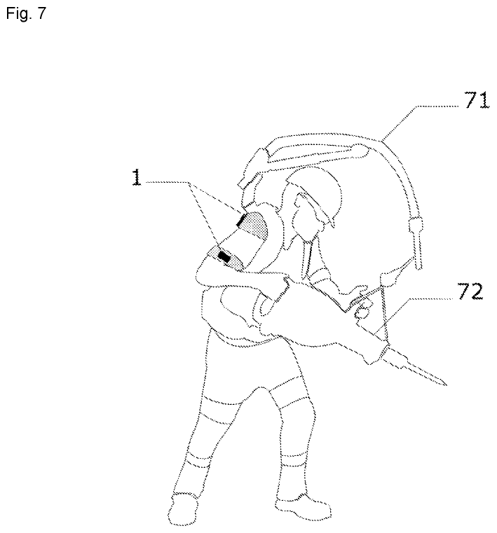

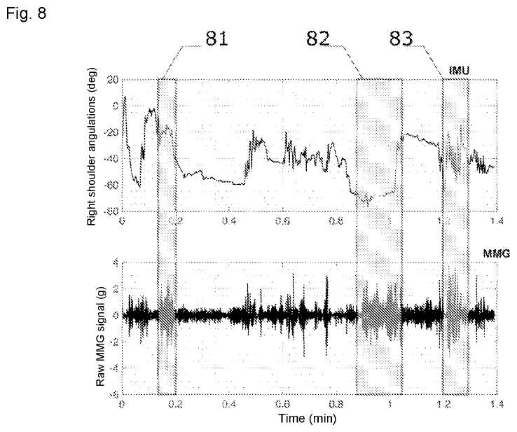

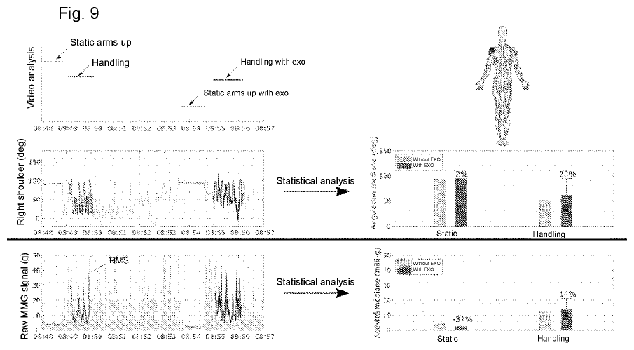

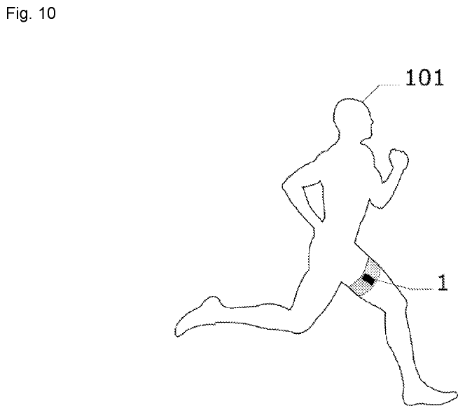

In a particular mode of operation relating to physical ergonomics, the method of the present disclosure as well as the associated measurement system may be used to produce ergonomic ratings at a workstation or equipment item. Take the example of a worker, illustrated in FIG. 7, wearing an exoskeleton 71 in order to facilitate wearing a portable electric instrument 72. The movements and the vibratory activity are measured at the right shoulder and the right arm by the measurement system 1 to determine if the exoskeleton is beneficial to the health and safety of the worker (correction of postures, load distribution carried at arm's length, etc.). A typical example of collected data is shown in FIG. 8. The periods of strong muscular activity 81, the uncomfortable postures inducing excessive static efforts 82 can clearly be seen, as well as the dynamic forces during rapid movements 83. It is possible from this information to deliver a fully automated and objective ergonomic rating by comparing the situations before and after wearing the exoskeleton. Indeed, an alteration of the articular flexion angles of the arm may be the cause of a decrease or an increase in the RMS amplitude of the MMG signal (indicator of the deployed force), thus reflecting a different mobilization of the joints depending on whether or not operators are wearing the exoskeleton. Statistical analysis of the differences in the means between the situations without and the situations with wearing of the exoskeleton allows a map to be drawn up of the biomechanical risk as shown in FIG. 9.

Another innovative mode of operation of this disclosure resides in the use of movement data to segment the physical activity into different states (maintaining a position, recognizing cyclic gestures, etc.). This segmentation allows determination of the optimum acquisition parameters of the MMG sensor (acquisition window, filter template and sampling) in order to analyze the impact on the muscles of a gesture or a series of well-defined gestures. This method is used, in particular, for the segmentation of the squat activity by Woodward in [22]. The microcontroller of the measurement node calculates the RMS amplitude level to detect the stress level on the muscle, and gives an estimate of the PSD at each acquisition. The data extracted from the MMG sensor is retransmitted to the receiving station, which determines a time-frequency representation of the activity. This method allows correlation of gestures or a very precise series of gestures with vibratory signatures of muscular activity. The segmentation may be done a posteriori by manual action or automatically by the receiving station owing to gesture recognition methods and machine learning techniques.

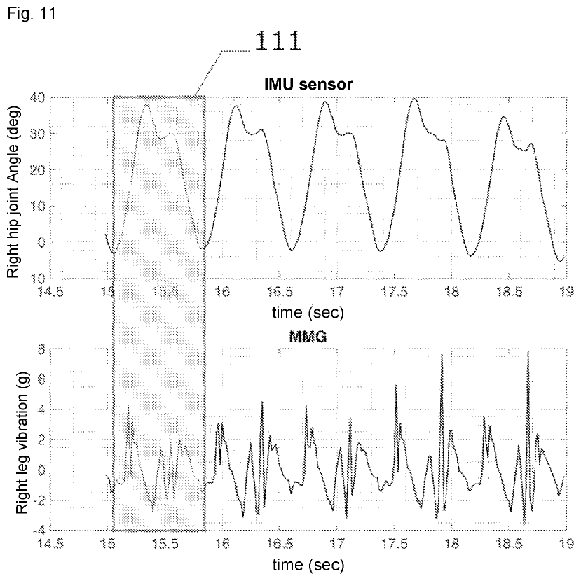

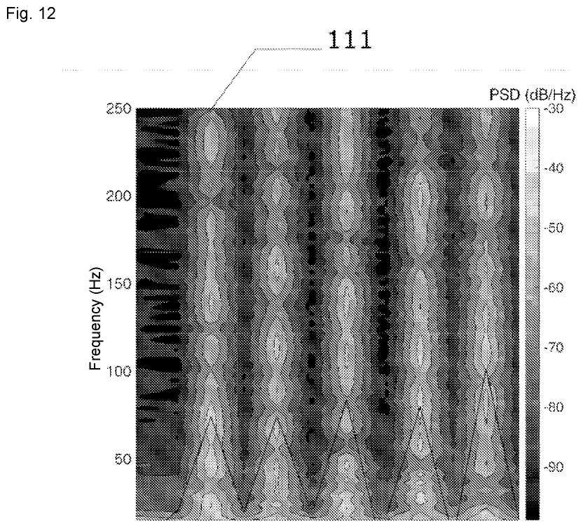

This mode of operation may find applications in the sports field, where an athlete seeks to develop his performance by perfecting technical gestures. In the example of FIG. 10, a runner 101 is equipped with the measurement system 1 in order to analyze the muscular fatigue produced by repeated gestures such as stride. The IMU and MMG signals typical of the joint of the right hip are presented in FIG. 11, in which the stride is characterized by the pattern in segment 111. The duration of this pattern (less than 1 second) allows determination of a window of acquisition for post-processing of the MMG signal. The measurement node proceeds to estimate the PSD, which is then processed by the receiving station to provide the STFT representation of FIG. 12. The power spectral density PSD is estimated by the Burg or Yule-Walker algorithm and may be observed over a frequency band between 20 Hz and 250 Hz. Each vertical line represents an acquisition with its frequency signature, that is, the distribution of the signal energy over the frequency band of interest. It is thus possible to observe the vibratory signature of the segment 111 characterizing the stride and to observe drifts over time of this signature, notably via the MPF, in order to prevent fatigue of the runner and optimize his training. Indeed, the variation of the MPF over time reflects the change in the muscle fiber recruitment strategy, in particular, by the ratio between the high- and low-frequency components of the MMG signal. High frequencies are caused by fast twitch fibers, while low frequencies are caused by slow twitch fibers. A drop in the ratio between high and low frequency (and, therefore, a decrease in MPF) over a long period allows peripheral fatigue to be objectified. Thus, when fatigue sets in, the fast fibers will tend to block and will then be partially supplemented by the activation of the slow fibers. Conversely, the phases of intense contractions for short periods of time will mobilize more fast muscle fibers and, therefore, increase the MPF.

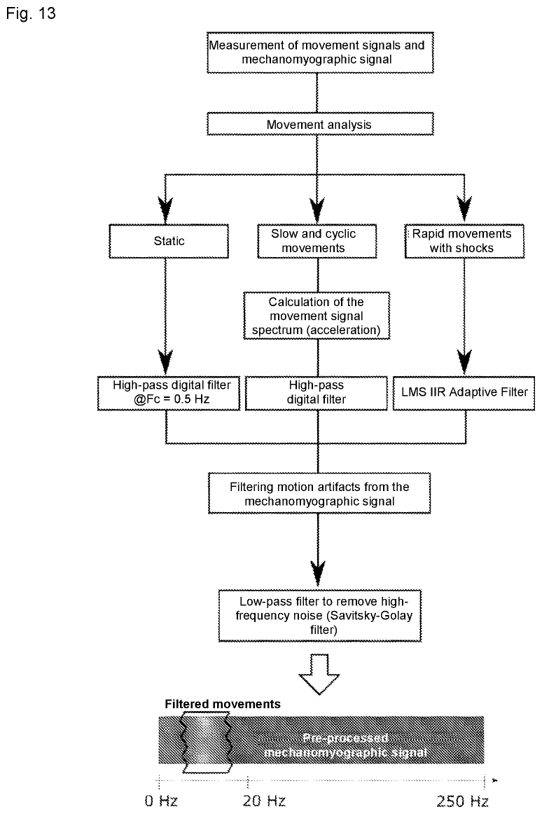

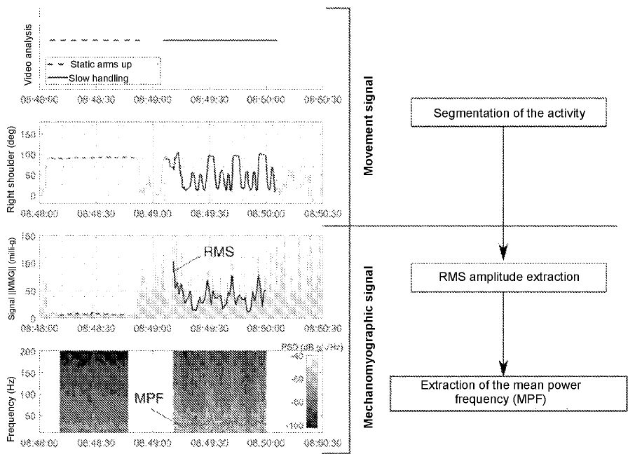

A description will now be given, with reference to FIG. 13, of a particular example of a method for obtaining a quality mechanomyographic signal used for the present disclosure.

The mechanomyographic signal (MMG), obtained by a 3-axis accelerometer, can detect changes in the behavior of the muscular activity due to fatigue and the intensity of the effort.

Filtering motion artefacts is a real challenge, since they will have changing characteristics related to the subject's activity. Indeed, to analyze the quadriceps during a typical walk, the movements of the leg have a frequency around 1 Hz, and reach up to 4 Hz during a fast run. In addition, the shocks transmitted by the impact of the foot will in turn transmit vibrations along the leg with a spectrum comprising components up to 20 Hz, to then be attenuated by the abdominal-lumbar belt.

It is, therefore, necessary to analyze the movement signals in order to choose the pre-processing operations to be applied to the mechanomyographic signal. The movement signals, coming from the inertial units, allow identification of static postures, sudden gestures, or pauses between different series of movements and will thus isolate “sequences” in the mechanomyographic signal. The identification of these sequences is the result of a physical activity segmentation operation that will facilitate the choice of signal processing operations to be applied to the mechanomyographic signal (MMG). Thus, with reference to FIG. 14, from the processing of the movement signal, a segmentation of the activity is carried out. Then, from the processing of the MMG signal, an extraction of the RMS amplitude and an extraction of the mean power frequency MPF are performed.

Using video may be practical to improve the quality of the segmentation in the event that it is done in post-processing and manually. Nevertheless, training algorithms through the use of data from inertial units and videos makes it possible to consider automating the segmentation of movement for real-time analyses.

In the example of a static posture, a digital 3-pole high-pass Butterworth filter with a cutoff at 0.5 Hz is a good candidate for processing the mechanomyographic signal.

Other types of filters may be used, such as Chebyshev or elliptical filters, which have steeper slopes in the rejected band to the detriment of ripples in the pass and/or rejected band.

In the case of slow movements, cycles and in the absence of shocks (i.e., impacts on the ground), calculating the spectrum of the acceleration signal allows identification of a narrow frequency band that can then be filtered by a digital 5-pole Butterworth passband filter.

In a last case with movements of variable dynamics and causing possible shocks, the acceleration signal of the IMU sensor may be used in an adaptive filtering process, for example, using the LMS (Least Mean Square) algorithm. Another technique employing a multi-resolution analysis (MRA) by applying 7-level Daubechies wavelets “db6” allows reconstruction of the mechanomyographic signal deprived of the components linked to the movement.

Applying a low-pass filter also cuts out high-frequency noise. A cutoff between 200 Hz and 250 Hz is ideal for analyzing the mechanomyographic signal. Using a Butterworth low-pass digital filter or a Savitzky-Golay filter are common practices in today's art.

Of course, the disclosure is not limited to the examples that have just been described, and many other embodiments may be envisaged without departing from the scope of the present disclosure.

### REFERENCES

1. M. B. I. Reaz, M. S. Hussain, and F. Mohd-Yasin. Techniques of EMG signal analysis: Detection, processing, classification and applications. Biological Procedures Online, 8(1): 11 35, March 2006.

2. W. H. Wollaston. On the duration of muscular action. Philos. Trans. R. Soc. London, pages 1-5, 1810.

3. F. V. Brozovich and G. H. Pollack. Muscle contraction generates discrete sound bursts. Biophysical Journal, 41(1): 35-40, 1983

4. N. Babault et al. “Neuromuscular fatigue development during maximal concentric and isometric knee extensions.” In: Journal of Applied Physiology 100.3 (March 2006), pp. 780-785.

5. G. Träger et al. “Comparison of phonomyography, kinemyography and mechanomyography for neuromuscular monitoring.” In: Canadian Journal of Anesthesia/Journal canadien d'anesthésie 53.2 (February 2006), pp. 130-135.

6. J. Marusiak et al. “Electromyography and mechanomyography of elbow agonists and antagonists in Parkinson's disease.” In: Muscle & Nerve 40.2 (August 2009), pp. 240-248.

7. A. Islam et al. “Mechanomyography Sensors for Muscle Assessment: a Brief Review.” In: Journal of Physical Therapy Science 24.12 (2012), pp. 1359-1365.

8. X. Robert-Lachaine, H. Mecheri, C. Lame, and A. Plamondon. Effect of local magnetic field disturbances on inertial measurement units accuracy. Applied Ergonomics 63: 123-132, September 2017.

9. P. Plantard. “Objectivation et standardisation des évaluations ergonomiques des postes de travail à partir de données Kinect.” [Objectification and standardization of ergonomic assessments of workstations based on Kinect data.] PhD thesis. July 2016.

10. Biosleeve human-machine interface. May 2013. URL: https://patents.google.com/patent/US20130317648A1/en.

11. Wearable Physiological Sensor System for Training and Therapeutic Purposes. April 2017. URL: https://patents.google.com/patent/US20170312576A1/en.

12. Systems, articles, and methods for gesture identification in wearable electromyography devices. December 2014. URL: https://patents.google.com/patent/US20150169074.

13. Methods and devices for combining muscle activity sensor signals and inertial sensor signals for gesture-based control. February 2014. URL: https://patents.google.com/patent/US20140240103.

14. Biomechanical activity monitoring. May 2015. URL: https://patentscope.wipo.int/search/en/detail.jsf?docid=W02015063520.

15. Real-time assessment of absolute muscle effort during open and closed chain activities. February 2010. URL: https://patents.google.com/patent/US20110196262A1/en.

16. J. Laine and D. Mougenot. “Benefits of MEMS Based Seismic Accelerometers for Oil Exploration.” In: TRANSDUCERS 2007—2007 International Solid-State Sensors, Actuators and Microsystems Conference. IEEE, 2007, pp. 1473-1477.

17. M. Baillargeon and L. Patry. “Les troubles musculo-squelettiques du membre supérieur reliés au travail: définitions, anatomie fonctionnelle, mécanismes physiopathologiques et facteurs de risque” [Work-related upper limb musculoskeletal disorders: definitions, functional anatomy, pathophysiological mechanisms and risk factors] In: Regie regionals de la santé et des services sociaux de Montréal-Centre. Direction de la santé publique (2003). URL: http://www.bdsp.ehesp.fr/Base/508934/.

18. MTw Awinda—Products—Xsens 3D motion tracking. URL: https://www.xsens.com/products/mtw-awinda/.

19. ADXL355 Datasheet and Product Info|Analog Devices. URL: https://www.analog.com/en/products/adx1355.html.

20. A. M. Sabatini. “Quaternion-based strap-down integration method for applications of inertial sensing to gait analysis.” In: Medical & Biological Engineering & Computing 43.1 (February 2005), pp. 94-101.

21. M. Ibitoye et al. “Mechanomyographic Parameter Extraction Methods: An Appraisal for Clinical Applications.” In: Sensors 14.12 (December 2014), pp. 22940-22970.

22. R. B. Woodward et al. “Segmenting Mechanomyography Measures of Muscle Activity Phases Using Inertial Data.” In: Scientific Reports 9.1 (December 2019), p. 5569.

1. A system for analyzing biomechanical activity of a human subject and exposure to a biomechanical risk factor in a context of physical activity, comprising:
a. means for collecting vibratory signals, attached to one or more first body segments of the human subject, a measurement of which reflects local muscular activity;
b. means for collecting signals representing movement of the human subject, a measurement of which reflects orientation and movement of one or more second body segments in 2 or 3 dimensions;
c. means for processing these signals so as to extract therefrom indicators representative of intensity of the of biomechanical stress;
d. means for detecting a drift of the vibratory signals with respect to a frame of reference of the vibratory behavior of-said muscle(s) in the context of physical activity; and
e. means for predicting a physiological break time necessary for the muscles of the human subject to recover their reference vibratory behavior.

2. The system of claim 1, further comprising means for processing the detected drift and producing biomechanical indicators therefrom.

3. The system of claim 1, wherein the means for collecting the movement signals comprise an inertial unit IMU (Inertial Measurement Unit).

4. The system of claim 3, wherein the inertial unit IMU is integrated together with the muscular activity sensor means to obtain co-localized measurements at a body segment of the human subject.

5. The system of claim 3, wherein the inertial unit IMU is is configured to measure rectilinear accelerations relative to three axes and rotations relative to three axes.

6. The system of claim 1, wherein the means for collecting the movement signals further comprise a three-axis magnetometer to determine the orientation of a body segment of the human subject with respect to the Earth's magnetic North.

7. The system of claim 1, wherein the means for sensing muscular activity comprise an MMG (mechanomyographic) accelerometer arranged to generate a mechanomyographic signal.

8. The system of claim 1, wherein the analysis system implements comprises a plurality of measurement nodes configured to be firmly attached to body segments of a human subject.

9. The system of claim 8, wherein a measurement node of the plurality of measurement nodes comprises means of communication with a receiving station.

10. The system of claim 9, wherein the means of communication implement a Bluetooth Low Energy (BLE) communication protocol.

11. A method for analyzing the biomechanical activity of a human subject and exposure to a biomechanical risk factor in a context of physical activity, implemented using a system according to claim 1, comprising:
a. collecting vibratory signals by a vibration sensor attached to one or more first body segments of the human subject, a measurement of which reflects the local muscular activity;
b. collecting signals representing movement of the human subject, a measurement of which reflects orientation and movement of one or more second body segments in 2 or 3 dimensions;
c. processing these signals to extract indicators representative of intensity of biomechanical stress, this signal processing generating a frequency signature of the biomechanical activity;
d. detecting a drift of the vibratory signals with respect to a frame of reference of the vibratory behavior of muscle(s) in the context of physical activity; and
e. predicting a physiological break time necessary for the muscles of the human subject to recover their reference vibratory behavior.

12. The method of claim 11, further comprising collecting the context or a scene in which the human subject is evolving by optical collection means, wherein the method further comprises processing this-the context or the scene so as to generate information on a posture and a gesture of the human subject in correlation with the vibratory behavior signals.

13. The method of claim 12, further comprising segmenting the physical activity of the human subject into specific and/or repetitive tasks or groups of tasks and correlating them with the muscular activity signals so as to estimate condition of the muscle and its drift over time.

14. The method of claim 11, further comprising a step of generating, from a set of biomechanical characterizations of muscular activity obtained for a given human subject, an individualized biomechanical risk frame of reference for this human subject.

15. The method of claim 14, wherein the step of generating an individualized biomechanical risk frame of reference implements a machine learning technique.

16. The method of claim 11, further comprising performing a cross-analysis of the movement and muscular activity signals so as to deliver information on performance and health of the human subject during a repeated muscular activity.

17. The method of claim 11, further comprises processing movement data and muscular activity data so as to recommend a personalized arrangement of physiological breaks so that muscular tissues of the human subject return to their rested state in a metabolic and mechanical sense after an effort.
